(19) United States
(12) Patent Application Publication
(10) Pub. No.: US 2023/0002821 A1
(43) Pub. Date: Jan. 5, 2023

(54) HIGH-THROUGHPUT DETECTION METHOD FOR RARE MUTATION OF GENE

(71) Applicant: SUZHOU SMK GENE TECH LTD., Jiangsu (CN)

(72) Inventors: Weishi YU, Jiangsu (CN); Mengmeng LIANG, Jiangsu (CN); Feng YANG, Jiangsu (CN)

(21) Appl. No.: 17/783,365

(22) PCT Filed: May 28, 2020
(86) PCT No.: PCT/CN2020/092900

§ 371 Date: Jun. 8, 2022

(30) Foreign Application Priority Data
Dec. 20, 2019 (CN) 201911330414.X

(51) Int. Cl. C12Q 1/6869; C12Q 1/686; C12N 15/10; C12Q 1/6855

(52) U.S. Cl. CPC C12Q 1/6869; C12Q 1/686; C12N 15/1065; C12Q 1/6855

(57) ABSTRACT

The present invention belongs to the fields of biomedical technology and molecular diagnosis. Disclosed is a high-throughput detection method for a rare mutation of a gene, comprising: designing specific probes; connecting Y-shaped universal linkers to a test DNA subjected to fragmentation processing, and performing amplification and enrichment of a target site by universal sequence combination of the specific probes and the linkers; performing genomic sequence alignment on sequences to be sequenced; sorting and analyzing said sequences at the same starting and ending positions, and filtering sequencing errors; and after the data filtering, the sequencing depth count of a reference allele of the target site being a, and the sequencing depth count of other alleles being b, and thus the actual mutation ratio of the site being b/(a+b). This technique can perform, by DNA fragmentation, universal linker connection, multiplex PCR amplification of specific primers and linker sequence primers, and high-throughput high-depth sequencing, enrichment and parallel sequencing on a plurality of sites to be tested.

### TECHNICAL FIELD

The present disclosure belongs to the fields of biomedical technology and molecular diagnostics and, in particular, to a high-throughput detection method for rare mutation of gene.

### BACKGROUND

With an increasingly deep understanding of a molecular basis of genetic disorders, a technical ability to detect a rare mutation in a specific gene continues to be improved. The rare mutation refers to a relatively rare mutant-type DNA sequence among a large number of wild-type DNA sequences, such as a small amount of tumor mutant gene DNA contained in the blood of a tumor patient, a small amount of tumor mutant DNA remaining in the blood of a cancer patient after treatment, a small amount of fetal DNA contained in the blood of a pregnant woman, a small amount of different genetic traits which are chimeric or mixed in a chimera and a drug-resistant mutation of bacteria or viruses occurring at an initial stage. All the above belong to a category of the rare mutation, and a rare mutation in a narrow sense generally refers to a point mutation. The above mutations are often associated with a disease, either as a direct cause of an attack of a certain disease or as an early sign or an important biomarker of an attack of a certain disease. Therefore, the rare mutation is closely related to human health, and the detection of the rare mutation is of very positive significance in non-invasive prenatal diagnosis, early screening of diseases, disease prognosis and treatment evaluation. Many methods are used for detecting the mutation, but most of the reported methods are limited to a qualitative detection of the mutation and an accurate quantitative detection cannot be performed, where a high-throughput method for quantitatively detecting the rare mutation is especially rarer. Several main detection methods at present are briefly described below.

### 1. Measurement Methods Based on Capillary Electrophoresis Detection

One method is a detection technique based on Sanger sequencing. The method mainly includes: separating and purifying the DNA of a sample to be detected, designing a primer for a site to be detected to perform amplification, and then performing direct sequencing. Whether a rare mutation exists may be determined by determining whether a trace allele different from a wild-type gene exists in a sequencing result. Another method is a method based on terminated extension of a fluorescently labeled specific nucleotide. The method mainly includes: designing an amplification primer for a site to be detected to perform polymerase chain reaction (PCR) amplification on an object fragment, designing a specific extension primer for the site to be detected, and then selectively replacing a corresponding nucleotide in a single deoxynucleotide (dNTP) with one nucleotide in a fluorescently labeled dideoxynucleotide (ddNTP) according to sequence characteristics of the site to be detected. When mutant-type DNA sequence exists in DNA, the extension is terminated at a position of several bases downstream of a target site instead of a position of the target site, and a trace signal is detected through capillary electrophoresis. Defects of these methods are inaccurate detection results and relatively low detection sensitivity when the capillary electrophoresis detection has a relatively high background.

### 2. Detection Method Based on Amplification Refractory Mutation System Technology

Amplification refractory mutation system (ARMS), also known as allele specific amplification (ASA), is a method firstly developed by Newton et al. to detect a known mutation. A basic principle is that if a base at a 3′-end of a primer is not complementary to a template base, the primer cannot be extended with a general heat-resistant DNA polymerase. Therefore, three primers are designed according to a known point mutation, a base at a 3′-end of each of the three primers is separately complementary to a mutant template base and a normal template base, so as to distinguish a template with a certain point mutation from a normal template. At present, this technology has become one of the important methods for an individualized molecular detection of tumors in the world. The disadvantages of this method are that this method cannot perform a quantitative detection on a rare mutation and is not suitable for detecting multiple sites simultaneously.

### 3. Detection Method Based on Digital PCR

At the end of the 20th century, Vogelstein et al. proposed the concept of a digital PCR (dPCR). One sample is divided into dozens to tens of thousands of parts which are assigned to different reaction units, where each unit contains one or more copied target molecules (DNA templates), PCR amplification is separately performed on the target molecules in each reaction unit, and after the amplification, a statistical analysis and quantification are performed on fluorescence signals of each reaction unit. This method requires a dPCR platform, increasing an operation difficulty and a detection cost.

### SUMMARY

The present application provides a high-throughput detection method for rare mutation of a gene. Through DNA fragmentation, universal adapter connection, multiplex PCR amplification with specific primers and adapter sequence primers and high-throughput and high-depth sequencing, this technology can perform parallel sequencing on multiple sites to be detected, align and splice sequencing sequences, and analyze and remove a (false positive) sequence with a sequencing error by specifically splicing the sequences, improving the accuracy of a quantitative detection and analysis of a rare mutation.

To solve the above technical problems and achieve the above technical object, the present application adopts the following technical solution: a high-throughput detection method for rare mutation of gene. The method includes the steps below:

designing a specific probe: a pair of a forward-strand probe and a reverse-strand probe is designed for each site to be detected, where in each pair of probes, the forward-strand probe is located on a positive strand of a gene sequence and the reverse-strand probe is located on a negative strand of a genome sequence;

constructing a genomic library: DNA to be detected is fragmented and ligated to a Y-type universal adapter, and PCR amplification is performed with a forward universal primer and a reverse universal primer so that the genomic library is constructed;

amplifying the genomic library: the forward-strand probe and the reverse universal primer form an amplification primer combination 1, the reverse-strand probe and the forward universal primer form an amplification primer combination 2, and the constructed genomic library of the DNA to be detected is amplified through the two combinations, separately;

classifying samples of sequencing sequences: the primer combination 1 and the primer combination 2 are amplified with PCR primers, a product of a second round of PCR amplification is subjected to high-throughput pair-ended sequencing, and sequencing data is analyzed so that the samples of the sequencing sequences are classified;

performing genome sequence alignment: sequences obtained through the sequencing are assigned to corresponding samples according to tag sequences and then to amplification products of corresponding gene fragments according to a base composition of each sequence; analyzing sequencing data: sequencing sequences with the same start position and end positions are classified and analyzed, a statistical count of such sequences is N, a certain base type whose count is below 10%*N at a target site is regarded as a sequencing error and filtered, after the filtration, a sequencing depth of an allele of each target site is counted, a sequencing depth of a reference allele of the target site is counted as a, a sequencing depth of another allele (mutation) of the target site is counted as b, and a true mutation proportion of the target site is b/(a+b).

As an improved technical solution of the present application, a sequence of a moiety at a 5′-end of each of the forward-strand probe or the reverse-strand probe is a universal sequence consistent with a last labeled PCR amplification primer.

As an improved technical solution of the present application, a moiety at a 3′-end of each of the forward-strand probe or the reverse-strand probe is a sequence specifically binding to an upstream region of a moiety at a 5′-end where the site to be detected is located.

As an improved technical solution of the present application, a distance between a 3′-end of a specific binding sequence and the site to be detected is 2-100 bp.

As an improved technical solution of the present application, the specific probe has a length of 18-36 bp.

As an improved technical solution of the present application, the specific probe has a length of 20-27 bp.

As an improved technical solution of the present application, each of the forward universal primer and the reverse universal primer contains a sequence the same as or reversely complementary to a bifurcated end of the Y-type universal adapter so that each DNA molecule, both ends of which are ligated to the universal adapter, is subjected to the PCR amplification.

As an improved technical solution of the present application, after fragmented, the DNA has a length of 200-1000 bp.

As an improved technical solution of the present application, during the construction of the genomic library, the number of cycles of the PCR amplification is 6-12.

As an improved technical solution of the present application, when the product of the second round of PCR amplification is subjected to the high-throughput pair-ended sequencing, an average sequencing depth is greater than 50000×.

The high-throughput detection method for rare mutation of gene according to the present disclosure mainly has the advantages below.

1. Detection throughput is improved: one reaction can detect dozens to thousands of sites simultaneously.

2. Detection cost is reduced: the high-throughput method is applied to a non-specific detection platform and does not require the input of an additional device, and one detection reaction can finish analyzing hundreds of thousands of gene fragments so that the detection cost of a single gene fragment is significantly reduced.

3. The high-throughput method is applied flexibly: a detection system can be quickly established for any object gene fragment to be detected.

4. Accuracy is improved: digital counting is used for quantification, and a specific analysis method is used for reducing an effect of the detection background of the sequencing error (the false positive) so that the accuracy is significantly improved.

5. Detection sensitivity is improved: sequence identification and digital counting for quantification using the sequencing on a single-molecule amplification product can significantly improve the sensitivity.

### BRIEF DESCRIPTION OF DRAWINGS

FIG. 1 is a schematic diagram of a design of a forward-strand probe and a reverse-strand probe for a site to be detected.

FIG. 2 is a schematic diagram of a structure and a sequence of a Y-type universal adapter.

FIG. 3 is a schematic diagram of a sequencing analysis.

### DETAILED DESCRIPTION

To illustrate the objects and technical solutions of embodiments of the present disclosure more clearly, the technical solutions in the embodiments of the present disclosure are described clearly and completely below in conjunction with drawings in the embodiments of the present disclosure. Apparently, the described embodiments are part, not all, of embodiments of the present disclosure. Based on the described embodiments of the present disclosure, all other embodiments obtained by those of ordinary skill in the art without creative work are within the scope of the present disclosure.

It is to be understood by those skilled in the art that unless otherwise defined, all terms (including technical terms and scientific terms) used herein have the same meanings as those commonly understood by those of ordinary skill in the art to which the present disclosure pertains. It is also to be understood that terms such as those defined in a general dictionary are to be construed as having meanings consistent with the meaning in the context of the existing art and are not to be interpreted in an idealized or overly formal sense, unless as defined herein.

A technical solution of a high-throughput detection method for rare mutation of gene is described below.

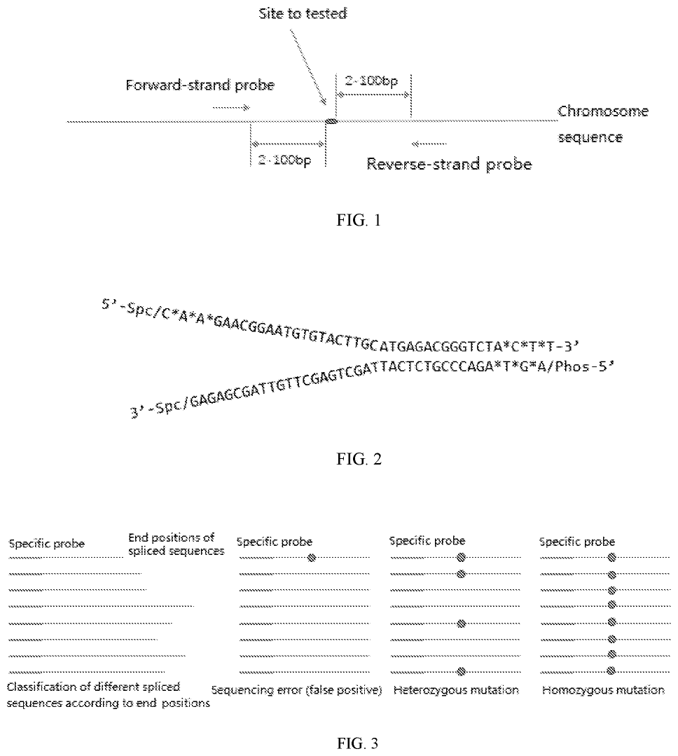

(1) A pair of a forward-strand probe and a reverse-strand probe (shown in FIG. 1) is separately designed for multiple sites to be detected, where each pair of probes are located on a positive strand and a negative strand of a genome sequence, separately, a sequence of a moiety at a 5′-end of each probe is a universal sequence consistent with a last tag-labeled PCR amplification primer, and a moiety at a 3′-end of each probe is a sequence specifically binding to an upstream region of a 5′ where a site to be detected is located. A distance between a 3′-end of a specific binding sequence and the site to be detected is 2-100 bp. The specific probe has a length of preferably 18-36 bp, more preferably 20-27 bp.

(2) DNA to be detected is fragmented by a physical method (such as ultrasound) or a chemical method (such as random enzyme digestion or a transposase). The fragment length of DNA after treatment is preferably 200-1000 bp. The fragmented DNA to be detected is ligated to a Y-type universal adapter (shown in FIG. 2), and PCR amplification is performed with a forward universal primer and a reverse universal primer so that a genomic library is constructed. Each of the forward universal primer and the reverse universal primer contains a sequence the same as or reversely complementary to a bifurcated end of the Y-type universal adapter so that each DNA molecule, both ends of which are ligated to the universal adapter, may be subjected to the PCR amplification to obtain a whole genomic library. Preferably, a number of cycles of the amplification is 6-12.

(3) The forward-strand probe in (1) and the reverse universal primer in (2) form an amplification primer combination 1, the reverse-strand probe in (1) and the forward universal primer in (2) form an amplification primer combination 2, and the constructed genomic library of the DNA to be detected is amplified through the two combinations, separately. As described in (2), the constructed whole genomic library of the DNA to be detected contains a sequence structure of the universal adapter, and one universal amplification primer of the adapter and one specific probe designed for a target site can enrich and amplify fragments containing the target site in the whole genomic library.

(4) Amplification products of the primer combination 1 and the primer combination 2 are mixed in equal amounts, and the mixture is amplified by a pair of PCR primers matched with sequencing primers of a second-generation sequencing platform. Generally, each of the PCR primers has a tag sequence with a length of several to dozens of bases, and amplification products from different samples may be amplified by PCR primers with different tag sequences so that the amplification products from different samples may be mixed together and in data of the subsequent high-throughput sequencing, sequences obtained through the sequencing may be assigned to different samples according to the tag sequences.

(5) A product of a second round of PCR amplification is subjected to high-throughput pair-ended sequencing. The sequencing may have a read length of PE150-300 bp, and an average sequencing depth is preferably greater than 50000X.

(6) The sequencing data is analyzed, and samples of sequencing sequences are classified. Genome sequence alignment is performed as follows: the sequences obtained through the sequencing are assigned to corresponding samples according to the tag sequences and then to amplification products of corresponding gene fragments according to a base composition of each sequence. As described in (3), one universal amplification primer of the adapter and one specific probe designed for the target site are used for amplification so that sequencing sequences containing the target site may be obtained. As shown in FIG. 3, the sequencing sequences start from the same specific probe and have different end positions from each other due to different breakpoint positions of random fragmentation. Sequencing sequences with the same start position and end positions are classified and analyzed, a statistical count of such sequences is N, a certain base type whose count is below 10%*N at the target site is regarded as a sequencing error and filtered, after the filtration, a sequencing depth of an allele of each target site is counted, a sequencing depth of a reference allele of the target site is counted as a, a sequencing depth of another allele (mutation) of the target site is counted as b, and a mutation proportion of the target site is b/(a+b).

### EXAMPLE 1

A mutation proportion was detected for each of 46 single-nucleotide polymorphism (SNP) sites in simulated samples.

Probes were designed for the 46 SNP sites, and a pair of a forward-strand probe and a reverse-strand probe was designed for each site, separately. Information about probes and universal primers are shown in Table 1 (a sequence listing).

| SNP | primer_F | primer_R |
|---|---|---|
| rs4952008 | CCTACACGACGCTCTTCCGATCT | CCTACACGACGCTCTTCCGATCT |
| | GCAACTACAAGAGGCTGAATGC | GGAGAAATGAGAGGCCTGATG |
| rs17393536 | CCTACACGACGCTCTTCCGATCT | CCTACACGACGCTCTTCCGATCT |
| | AAAGCCCAGCACCTCTCACC | CCATTCCATTGTGCCCTGTG |
| rs6720163 | CCTACACGACGCTCTTCCGATCT | CCTACACGACGCTCTTCCGATCT |
| | TGTCTGAAGGCTGACTGAATAAT | ACAGAGCCCTCCCACAGAGG |
| | CAAC | |
| rs212758 | CCTACACGACGCTCTTCCGATCT | CCTACACGACGCTCTTCCGATCT |
| | TGCAAAAATTGAAGTTGTCAGCA | AAGGTTGGTAGGGGCCTGATT |
| | C | |
| rs12523576 | CCTACACGACGCTCTTCCGATCT | CCTACACGACGCTCTTCCGATCT |
| | TCCCAAGATTAATTGGTCCTCTC | ACTTAGGATGGACACAAAGCAGA |
| | A | A |
| rs2932778 | CCTACACGACGCTCTTCCGATCT | CCTACACGACGCTCTTCCGATCT |
| | TCCTGGGCCTGCTTCTTCAT | GCCAAGTTCTTACGGGCTGGT |
| rs549799068 | CCTACACGACGCTCTTCCGATCT | CCTACACGACGCTCTTCCGATCT |
| | CCTCCTCTCCCTCTGCGTTT | AAAGGGGCCTGCACCATTTA |
| rs4718001 | CCTACACGACGCTCTTCCGATCT | CCTACACGACGCTCTTCCGATCT |
| | CCTCATTTGTCCTGACCATGTCTC | ATCTCAGAGTCTCCTGGCTGGTG |
| rs6460282 | CCTACACGACGCTCTTCCGATCT | CCTACACGACGCTCTTCCGATCT |
| | GCTGGTGCTCTGCTAAGTATGCT | CAAAAGATGTTGAATGCCTGAAG |
| | C | C |
| rs6976912 | CCTACACGACGCTCTTCCGATCT | CCTACACGACGCTCTTCCGATCT |
| | CCCTCCCCTCGTTTCCTTTC | CTGTGCTGGGGTCATGGAA |
| rs716578 | CCTACACGACGCTCTTCCGATCT | CCTACACGACGCTCTTCCGATCT |
| | TTGTGTGGACCAAGCCACCT | TTGGGGCTCTCTACCATGGAC |
| rs10215330 | CCTACACGACGCTCTTCCGATCT | CCTACACGACGCTCTTCCGATCT |
| | TGCCAAGTTCCCAAGGACTAA | GCCACCTAGGTAACATTTAGCTAC |
| | | CA |
| rs41624 | CCTACACGACGCTCTTCCGATCT | CCTACACGACGCTCTTCCGATCT |
| | TTCACAGGCCACATCAAAAAGTC | TGGCACCTAGATTTTATTTACACTT |
| | | TATCT |
| rs11974016 | CCTACACGACGCTCTTCCGATCT | CCTACACGACGCTCTTCCGATCT |
| | AACAGCCATTTGGGTGAATTTT | GGATTAAAGAAACCATTGTATGTC |
| | | CTG |
| rs6946211 | CCTACACGACGCTCTTCCGATCT | CCTACACGACGCTCTTCCGATCT |
| | AACAACATAATGGGATTGTTGAG | CCCTCAAGAAGAAAAGGCATTGA |
| | G | |
| rs1027617 | CCTACACGACGCTCTTCCGATCT | CCTACACGACGCTCTTCCGATCT |
| | CCCTAACTTCCAGATCAATCACA | GGATCAGGAGACTCAAACACTAC |
| | C | AAT |
| rs7119071 | CCTACACGACGCTCTTCCGATCT | CCTACACGACGCTCTTCCGATCT |
| | GGGGCCAAAGTAGGTGAGGA | CACCTTTCATCTTCATGGTCTTCC |
| rs11635373 | CCTACACGACGCTCTTCCGATCT | CCTACACGACGCTCTTCCGATCT |
| | AGGACCCAGGCAGAATAGCC | GCTCTGGACGCTAGCCATGT |
| rs8032357 | CCTACACGACGCTCTTCCGATCT | CCTACACGACGCTCTTCCGATCT |
| | CAATGAGGCAGTCATTTGTGAGC | CAACAAAACAAGCATTTATTTCTG |
| | | CAA |
| rs58156069 | CCTACACGACGCTCTTCCGATCT | CCTACACGACGCTCTTCCGATCT |
| | GGCTGAGAGTCAAACCATCCTTT | GGGGTCCCTCTACAGGAACATT |
| | T | |
| rs8037958 | CCTACACGACGCTCTTCCGATCT | CCTACACGACGCTCTTCCGATCT |
| | TCCATTCTTTGGTTGGTTCCTCA | GAGCTGCATGAAATTTGGAGCTT |
| rs9962222 | CCTACACGACGCTCTTCCGATCT | CCTACACGACGCTCTTCCGATCT |
| | AACACAGCTCAGTCGTGGTTG | TGAGCTCCTTGGCATTCAGG |
| rs1011947 | CCTACACGACGCTCTTCCGATCT | CCTACACGACGCTCTTCCGATCT |
| | TGTGGCAGTGGCTGCAATAG | AAAGCCAGAGCCCTTTCTTGTC |
| rs3786343 | CCTACACGACGCTCTTCCGATCT | CCTACACGACGCTCTTCCGATCT |
| | TCTGGGTTTATCTACCTCAGAAG | CAAAAATGAAATGGATTCATCAGG |
| | GTG | A |
| rs8409 | CCTACACGACGCTCTTCCGATCT | CCTACACGACGCTCTTCCGATCT |
| | CGACAGGATGTCGTCGGAGAT | CTCCCACCCCAGAACATCTCATC |
| rs7256770 | CCTACACGACGCTCTTCCGATCT | CCTACACGACGCTCTTCCGATCT |
| | GGCTGTAGGCTCCGCATCTGTA | CAGCTGCAGTCCGTGGGTCT |
| rs55892736 | CCTACACGACGCTCTTCCGATCT | CCTACACGACGCTCTTCCGATCT |
| | CCTGCCAAGAAACAGAGACACA | GGACACTTACATCCCCATCTTGG |
| | A | |
| rs36086386 | CCTACACGACGCTCTTCCGATCT | CCTACACGACGCTCTTCCGATCT |
| | TCCTGATCACTCAGCCTGAAGAC | TGAAGGGTATGCCTGTCGTG |
| rs417987 | CCTACACGACGCTCTTCCGATCT | CCTACACGACGCTCTTCCGATCT |
| | CCAAGAAGGATAACTGATGGACT | AGCAGTTCCTGCTTGCCATG |
| | C | |
| rs4829106 | CCTACACGACGCTCTTCCGATCT | CCTACACGACGCTCTTCCGATCT |
| | TGATAATCTAATTTTGTTTGTGCT | AGCAGCTGGGACCTTTTTGC |
| | CCA | |
| rs5972594 | CCTACACGACGCTCTTCCGATCT | CCTACACGACGCTCTTCCGATCT |
| | TTGATATTCATCTTGGCACCCATA | CAGCAGTTTTATTCTCTTCACTGC |
| | | AA |
| rs1547017 | CCTACACGACGCTCTTCCGATCT | CCTACACGACGCTCTTCCGATCT |
| | TTCACGCACTTTCAGAAGTCCT | TTCATTTACTGGATTCAAGTTACA |
| | | GTCC |
| rs1554311 | CCTACACGACGCTCTTCCGATCT | CCTACACGACGCTCTTCCGATCT |
| | GTGCTGAAACAGAGACAGGTAA | CCTGATATCACTACTGCAGACAAA |
| | AAA | CTAAT |
| rs12556557 | CCTACACGACGCTCTTCCGATCT | CCTACACGACGCTCTTCCGATCT |
| | CACTCTGACAGTGAGGTTCTTGG | AGGGCATGTCATTGCCTCAAAG |
| rs5906428 | CCTACACGACGCTCTTCCGATCT | CCTACACGACGCTCTTCCGATCT |
| | ACTCCCCCGAAATGAACAGC | CCTGGAGAGCGCCATCTCTG |
| rs28727466 | CCTACACGACGCTCTTCCGATCT | CCTACACGACGCTCTTCCGATCT |
| | TGGAGACGCAGAGAAGAGAACG | CATTCGGGTAGAACGTTGTATTAC |
| | | AGT |
| rs4554617 | CCTACACGACGCTCTTCCGATCT | CCTACACGACGCTCTTCCGATCT |
| | GCAGGGAAAAGGCTCAGTCC | TCCAGAGCTTTGCAGTGTTCTTC |
| rs7889048 | CCTACACGACGCTCTTCCGATCT | CCTACACGACGCTCTTCCGATCT |
| | ATGGGCCAGGTTTTGAGCTG | CATGGAGGTGACGGGCTATC |
| rs6614542 | CCTACACGACGCTCTTCCGATCT | CCTACACGACGCTCTTCCGATCT |
| | TCCTTGGGCCTGAGAAGACC | AGGATCCACTCCCCCTACCC |
| rs12557717 | CCTACACGACGCTCTTCCGATCT | CCTACACGACGCTCTTCCGATCT |
| | ATGGGGAGTGGGTGGGTAATG | TGGGATTCCACGTATGTGTTTGC |
| rs871865 | CCTACACGACGCTCTTCCGATCT | CCTACACGACGCTCTTCCGATCT |
| | CTGCTGGATAACTTGGAGGTGCT | TGCATATACTGCAGAGACAAGCA |
| | | AA |
| rs5930669 | CCTACACGACGCTCTTCCGATCT | CCTACACGACGCTCTTCCGATCT |
| | CATTTTCAGCTCCTTTCAAACCT | TGGGCAATCATTTTGAACCAA |
| | G | |
| rs2180062 | CCTACACGACGCTCTTCCGATCT | CCTACACGACGCTCTTCCGATCT |
| | CCCCCTGGAATTTTGTAAAGC | GCCCGGTTCTTGGAGATGAG |
| rs2300912 | CCTACACGACGCTCTTCCGATCT | CCTACACGACGCTCTTCCGATCT |
| | GACGACACCTCCAGGTGCATTAG | TTCAACAGATGGAGCAAAGCCTT |
| | | A |
| rs1005303 | CCTACACGACGCTCTTCCGATCT | CCTACACGACGCTCTTCCGATCT |
| | CTCAGGCCCAGGGCTTACTC | TAACAAGGCAGCCAGAAGCA |
| rs5929877 | CCTACACGACGCTCTTCCGATCT | CCTACACGACGCTCTTCCGATCT |
| | GGAACAGGGAAAGTCAGTGGTG | TGTGCCTGGCCAAGAGATAC |

Simulated samples with different mutation proportions (0.1%, 0.5%, 1%) were configured. DNA was fragmented through random enzyme digestion, ligated to a universal adapter and amplified through universal primers. A mixed solution of forward-strand probes at each site and the adapter universal reverse primer were subjected to PCR amplification to obtain an amplification product 1, and a mixed solution of reverse-strand probes at each site and the adapter universal forward primer were subjected to the PCR amplification to obtain an amplification product 2. The two products were mixed and amplified through PCR primers compatible with an Illumina sequencing platform and having different tag sequences. Products of all the samples were mixed uniformly and subjected to sequencing in a PE150 mode on an Illumina sequencing instrument, and sequencing data was analyzed subsequently.

Experimental Process

(1) A wild-type sample and a mutant sample were subjected to concentration quantification, and standards were configured according to the proportions (0.1%, 0.5%, 1%).

(2) 500 ng of each standard was diluted to 50 uL with a DNA diluent, 10 uL of a mixed solution for fragmentation Smearase® Mix was added, and the system was reacted for 1 min at 4 ° C., for 10 min at 30 ° C. and for 20 min at 72 ° C. After the reaction, 30 uL of Ligation Enhancer, 5 uL of T4 DNA ligases and 5 uL of universal adapters were added, the system was reacted for 15 min at 20 ° C. and ligated to the universal adapter, and a reaction product was purified through a DNA purification magnetic bead kit according to a proportion of 1×.

(3) A mixed solution 1 of forward-strand probes mixed solution-adapter universal reverse primers mixed solution and a mixed solution 2 of reverse-strand probes mixed solution-adapter universal forward primers mixed solution were used, where each primer had a concentration of 2 uM. 2 uL of ligation and purification product was used as a template for a PCR, and 20 uL of the reaction system contained 10 uL of 2x HIFI multi PCR master mix, 2 uL of Pmixl for P1 or Pmix2 for P2, 2 uL of ligation and purification product and 6 uL of sterile water. A PCR procedure was as follows: the system was reacted for 2 min at 98 ° C., subjected to 32x (reacted for 20s at 96 ° C. and for 4 min at 60 ° C.) and hold at 10 ° C., P1 and P2 were mixed in an equal proportion, and the reaction product was purified through the DNA purification magnetic bead kit according to a proportion of 1.8×.

(4) PCR primers UNIPCRF/UDIRxxxx were used, which were compatible with the Illumina sequencing platform and had different tag sequences, and each primer had a concentration of 2 uM. 2 uL of ligation and purification product was used as a template for the PCR, and 20 uL of the reaction system contained 10 uL of 2× HIFI multi PCR master mix, 2 uL of Pmix, 2 uL of ligation and purification product and 6 uL of sterile water. A PCR procedure was as follows: the system was reacted for 2 min at 98 ° C., subjected to 12x (reacted for 10s at 98 ° C., for 30s at 60 ° C. and for 30s at 72 ° C.) and hold at 10 ° C.(5) The final products were subjected to the sequencing in the PE150 mode on the Illumina sequencing platform, and the sequencing data was analyzed subsequently.

(6) Reads of the sequencing were assigned to different samples according to the tag sequences, spliced sequences starting from the same specific probe were classified according to different end positions, a false positive sequence of the same type of sequences was filtered, and finally, different alleles at a target site were counted.

Sequences of the universal primers used in this example are shown below (sequence listing).

Adapter Universal Primer F
TCAGACGTGTGCTCTTCCGATCTCAAGAACGGAATGTGTACTTGC
Adapter Universal Primer R
TCAGACGTGTGCTCTTCCGATCTCTCTCGCTAACAAGCTCAGCTA
UNIPCRF
AATGATACGGCGACCACCGAGATCTACACTCTTTCCCTACACGAC
UDIR0001
CAAGCAGAAGACGGCATACGAGAT AACCGCGG
GTGACTGGAGTTCAGACGTG
UDIR0002
CAAGCAGAAGACGGCATACGAGAT GGTTATAA
GTGACTGGAGTTCAGACGTG
UDIR0003
CAAGCAGAAGACGGCATACGAGAT CCAAGTCC
GTGACTGGAGTTCAGACGTG

SNP sites of three samples and the results of sequencing depths of alleles thereof are shown in Table 2.

| | | | | S1 | | S2 | |
|---|---|---|---|---|---|---|---|
| SNP | hg19 | Ref | Alt | Refdepth | Altdepth | Refdepth | Altdepth |
| rs417987 | chrX: 31071281 | T | C | 19831 | 20 | 21984 | 149 |
| rs4829106 | chrX: 31608475 | A | G | 64705 | 63 | 62425 | 436 |
| rs5972594 | chrX: 32548448 | G | A | 39549 | 52 | 72884 | 422 |
| rs1547017 | chrX: 32943611 | C | T | 33701 | 43 | 77189 | 371 |
| rs1554311 | chrX: 33603349 | C | T | 52888 | 112 | 43496 | 207 |
| rs12556557 | chrX: 47250485 | T | C | 48150 | 70 | 51010 | 252 |
| rs5906428 | chrX: 47415857 | A | G | 61839 | 79 | 39263 | 265 |
| rs28727466 | chrX: 48858802 | G | A | 40113 | 44 | 71764 | 339 |
| rs4554617 | chrX: 50203402 | A | C | 50084 | 55 | 79414 | 544 |
| rs7889048 | chrX: 50249685 | A | G | 57387 | 102 | 64861 | 314 |
| rs6614542 | chrX: 50311812 | C | G | 51563 | 84 | 69952 | 433 |
| rs12557717 | chrX: 133830383 | G | A | 58188 | 80 | 73764 | 376 |
| rs871865 | chrX: 133869349 | A | G | 45673 | 41 | 48970 | 275 |
| rs5930669 | chrX: 134069784 | C | T | 34452 | 55 | 66988 | 417 |
| rs2180062 | chrX: 135231388 | T | C | 77295 | 105 | 74346 | 424 |
| rs2300912 | chrX: 135281735 | T | C | 33242 | 57 | 45373 | 303 |
| rs1005303 | chrX: 136626238 | T | C | 30500 | 37 | 59700 | 252 |
| rs5929877 | chrX: 137128807 | A | G | 53291 | 108 | 30480 | 109 |
| rs8409 | chr19: 11319491 | G | A | 42684 | 38 | 62788 | 375 |
| rs7256770 | chr19: 11689911 | G | A | 41450 | 75 | 75705 | 509 |
| rs55892736 | chr19: 13109460 | A | G | 56138 | 87 | 36938 | 184 |
| rs36086386 | chr19: 13431397 | T | — | 72765 | 113 | 66145 | 285 |
| rs9962222 | chr18: 60965524 | G | A | 71385 | 135 | 45027 | 207 |
| rs1011947 | chr18: 61344012 | G | A | 39722 | 55 | 41626 | 255 |
| rs3786343 | chr18: 61647895 | C | T | 35988 | 61 | 37281 | 190 |
| rs11635373 | chr15: 48071698 | G | A | 73409 | 81 | 41730 | 157 |
| rs8032357 | chr15: 48618819 | A | G | 43497 | 56 | 64352 | 277 |
| rs58156069 | chr15: 49083577 | C | T | 57613 | 61 | 30266 | 193 |
| rs8037958 | chr15: 49279406 | A | G | 48157 | 97 | 44242 | 225 |
| rs1027617 | chr11: 16842787 | G | A | 75318 | 85 | 57320 | 314 |
| rs7119071 | chr11: 17572402 | A | C | 73571 | 117 | 63948 | 348 |
| rs4718001 | chr7: 63601237 | T | C | 67160 | 114 | 32227 | 142 |
| rs6460282 | chr7: 65691246 | T | C | 68320 | 65 | 46031 | 248 |
| rs6976912 | chr7: 66855183 | G | C | 63031 | 72 | 65137 | 326 |
| rs716578 | chr7: 67052528 | G | A | 40449 | 60 | 52102 | 311 |
| rs10215330 | chr7: 118872411 | G | A | 74321 | 98 | 58894 | 330 |
| rs41624 | chr7: 120435638 | T | C | 67882 | 91 | 64536 | 439 |
| rs11974016 | chr7: 121120235 | C | T | 65939 | 108 | 69430 | 455 |
| rs6946211 | chr7: 121554791 | C | T | 61642 | 120 | 32553 | 137 |
| rs12523576 | chr5: 68036921 | C | T | 41521 | 78 | 73686 | 300 |
| rs2932778 | chr5: 68530310 | T | C | 73010 | 146 | 32744 | 211 |
| rs549799068 | chr5: 71251209 | — | G | 65097 | 126 | 39325 | 227 |
| rs4952008 | chr2: 31172493 | C | T | 73730 | 127 | 45111 | 196 |
| rsl7393536 | chr2: 31240592 | T | C | 49893 | 70 | 31527 | 186 |
| rs6720163 | chr2: 31605411 | T | G | 69238 | 77 | 36184 | 183 |
| rs212758 | chr2: 32425982 | A | T | 37872 | 57 | 30940 | 169 |

| | | | Mutation Proportion Obtained | | |
|---|---|---|---|---|---|
| | S3 | | After Analyzing the Sequencing | | |
| SNP | Refdepth | Altdepth | S1 | S2 | S3 |
| rs417987 | 50071 | 1037 | 0.001008 | 0.006732 | 0.02029 |
| rs4829106 | 63013 | 826 | 0.000973 | 0.006936 | 0.012939 |
| rs5972594 | 50369 | 444 | 0.001313 | 0.005757 | 0.008738 |
| rs1547017 | 63208 | 544 | 0.001274 | 0.004783 | 0.008533 |
| rs1554311 | 39594 | 480 | 0.002113 | 0.004737 | 0.011978 |
| rs12556557 | 46134 | 891 | 0.001452 | 0.004916 | 0.018947 |
| rs5906428 | 47213 | 931 | 0.001276 | 0.006704 | 0.019338 |
| rs28727466 | 43784 | 867 | 0.001096 | 0.004702 | 0.019417 |
| rs4554617 | 56188 | 590 | 0.001097 | 0.006804 | 0.010391 |
| rs7889048 | 47210 | 510 | 0.001774 | 0.004818 | 0.010687 |
| rs6614542 | 35965 | 630 | 0.001626 | 0.006152 | 0.017215 |
| rs12557717 | 67103 | 1242 | 0.001373 | 0.005071 | 0.018173 |
| rs871865 | 39410 | 832 | 0.000897 | 0.005584 | 0.020675 |
| rs5930669 | 30687 | 350 | 0.001594 | 0.006186 | 0.011277 |
| rs2180062 | 76936 | 1331 | 0.001357 | 0.005671 | 0.017006 |
| rs2300912 | 37348 | 632 | 0.001712 | 0.006634 | 0.01664 |
| rs1005303 | 72725 | 590 | 0.001212 | 0.004203 | 0.008047 |
| rs5929877 | 31740 | 359 | 0.002023 | 0.003563 | 0.011184 |
| rs8409 | 34455 | 597 | 0.000889 | 0.005937 | 0.017032 |
| rs7256770 | 34192 | 691 | 0.001806 | 0.006679 | 0.019809 |
| rs55892736 | 56373 | 1010 | 0.001547 | 0.004957 | 0.017601 |
| rs36086386 | 72676 | 1490 | 0.001551 | 0.00429 | 0.02009 |
| rs9962222 | 48291 | 585 | 0.001888 | 0.004576 | 0.011969 |
| rs1011947 | 71602 | 1490 | 0.001383 | 0.006089 | 0.020385 |
| rs3786343 | 57016 | 639 | 0.001692 | 0.005071 | 0.011083 |
| rs11635373 | 69299 | 1338 | 0.001102 | 0.003748 | 0.018942 |
| rs8032357 | 69961 | 1428 | 0.001286 | 0.004286 | 0.020003 |
| rs58156069 | 64772 | 616 | 0.001058 | 0.006336 | 0.009421 |
| rs8037958 | 58647 | 1226 | 0.00201 | 0.00506 | 0.020477 |
| rs1027617 | 65651 | 1202 | 0.001127 | 0.005448 | 0.01798 |
| rs7119071 | 62171 | 914 | 0.001588 | 0.005412 | 0.014488 |
| rs4718001 | 55228 | 1122 | 0.001695 | 0.004387 | 0.019911 |
| rs6460282 | 39873 | 463 | 0.000951 | 0.005359 | 0.011479 |
| rs6976912 | 57397 | 856 | 0.001141 | 0.00498 | 0.014695 |
| rs716578 | 36910 | 488 | 0.001481 | 0.005934 | 0.013049 |
| rs10215330 | 78002 | 843 | 0.001317 | 0.005572 | 0.010692 |
| rs41624 | 65119 | 854 | 0.001339 | 0.006756 | 0.012945 |
| rs11974016 | 77068 | 833 | 0.001635 | 0.006511 | 0.010693 |
| rs6946211 | 41513 | 499 | 0.001943 | 0.004191 | 0.011878 |
| rs12523576 | 55672 | 446 | 0.001875 | 0.004055 | 0.007948 |
| rs2932778 | 76306 | 1618 | 0.001996 | 0.006403 | 0.020764 |
| rs549799068 | 69847 | 1034 | 0.001932 | 0.005739 | 0.014588 |
| rs4952008 | 39913 | 607 | 0.00172 | 0.004326 | 0.01498 |
| rsl7393536 | 60116 | 620 | 0.001401 | 0.005865 | 0.010208 |
| rs6720163 | 77822 | 1627 | 0.001111 | 0.005032 | 0.020479 |
| rs212758 | 33487 | 620 | 0.001503 | 0.005433 | 0.018178 |

After accurate quantification, the simulated samples are configured according to theoretical mutation proportions (0.1%, 0.5%, 1%). The sequencing error of the sequences with the same start position and end positions are filtered. The sequencing depth of the reference allele of the target site is counted as a, the sequencing depth of another allele (mutation) of the target site is counted as b, and the mutation proportion (b/(a+b)) is calculated through the count of a mutant allele. The detection results show that the mutation proportions of the simulated samples are consistent with the theoretical proportions.

The preceding are merely embodiments of the present disclosure, and the specific and detailed description thereof cannot be construed as limiting the scope of the present disclosure. It is to be noted that those of ordinary skill in the art can make a number of variations and improvements without departing from the concept of the present disclosure, and such variations and improvements are within the scope of the present disclosure.

What is claimed is:

1. A high-throughput detection method for rare mutation of gene, comprising:
designing a specific probe: a pair of a forward-strand probe and a reverse-strand probe is designed for each site to be detected, wherein in each pair of probes, the forward-strand probe is located on a positive strand of a gene sequence and the reverse-strand probe is located on a negative strand of a genome sequence;
constructing a genomic library: DNA to be detected is fragmented and ligated to a Y-type universal adapter, and polymerase chain reaction (PCR) amplification is performed with a forward universal primer and a reverse universal primer so that the genomic library is constructed;
amplifying the genomic library: the forward-strand probe and the reverse universal primer form an amplification primer combination 1, the reverse-strand probe and the forward universal primer form an amplification primer combination 2, the primer combination 1 and the primer combination 2 are mixed in equal amounts, the mixture is amplified with PCR primers, and a product of a second round of PCR amplification is subjected to high-throughput pair-ended sequencing, wherein the PCR primers used for the primer combination 1 and the primer combination 2 from different samples have different tag sequences, and the high-throughput pair-ended sequencing is defined as sequencing in a pair-ended sequencing mode and using a high-throughput sequencing platform;
performing genome sequence alignment: sequences obtained through the sequencing are assigned to corresponding samples according to the tag sequences and then to amplification products of corresponding gene fragments according to a base composition of each sequence;
analyzing sequencing data: sequencing sequences with the same start position and end positions are classified and analyzed, a statistical count of such sequences is N, a certain base type whose count is below 10%*N at a target site is regarded as a sequencing error and filtered, after the filtration, a sequencing depth of an allele of each target site is counted, a sequencing depth of a reference allele of the target site is counted as a, a sequencing depth of another allele of the target site is counted as b, and a true mutation proportion of the target site is b/(a+b).

2. The high-throughput detection method for rare mutation of gene according to claim 1, wherein a sequence of a moiety at a 5′-end of each of the forward-strand probe or the reverse-strand probe is a universal sequence consistent with a last labeled PCR amplification primer.

3. The high-throughput detection method for rare mutation of gene according to claim 1, wherein a moiety at a 3′-end of each of the forward-strand probe or the reverse-strand probe is a sequence specifically binding to an upstream region of a moiety at a 5′-end where the site to be detected is located.

4. The high-throughput detection method for rare mutation of gene according to claim 1, wherein a distance between a 3′-end of a specific binding sequence and the site to be detected is 2-100 bp.

5. The high-throughput detection method for rare mutation of gene according to claim 1, wherein the specific probe has a length of 18-36 bp.

6. The high-throughput detection method for rare mutation of gene according to claim 1, wherein the specific probe has a length of 20-27 bp.

7. The high-throughput detection method for rare mutation of gene according to claim 1, wherein each of the forward universal primer and the reverse universal primer contains a sequence the same as or reversely complementary to a bifurcated end of the Y-type universal adapter so that each DNA molecule, both ends of which are ligated to the universal adapter, is subjected to the PCR amplification.

8. The high-throughput detection method for rare mutation of gene according to claim 1, wherein after fragmented, the DNA has a length of 200-1000 bp.

9. The high-throughput detection method for rare mutation of gene according to claim 1, wherein during the construction of the genomic library, a number of cycles of the PCR amplification is 6-12.

10. The high-throughput detection method for rare mutation of gene according to claim 1, wherein when the product of the second round of PCR amplification is subjected to the high-throughput pair-ended sequencing, an average sequencing depth is greater than 50000×.


SEQUENCE LISTING (98 sequences)

SEQ ID NO: 1 - DNA, 45 bp, artificial sequence
  primer_F
cctacacgacgctcttccgatctgcaactacaagaggctgaatgc

SEQ ID NO: 2 - DNA, 44 bp, artificial sequence
  primer_R
cctacacgacgctcttccgatctggagaaatgagaggcctgatg

SEQ ID NO: 3 - DNA, 43 bp, artificial sequence
  primer_F
cctacacgacgctcttccgatctaaagcccagcacctctcacc

SEQ ID NO: 4 - DNA, 43 bp, artificial sequence
  primer_R
cctacacgacgctcttccgatctccattccattgtgccctgtg

SEQ ID NO: 5 - DNA, 50 bp, artificial sequence
  primer_F
cctacacgacgctcttccgatcttgtctgaaggctgactgaataatcaac

SEQ ID NO: 6 - DNA, 43 bp, artificial sequence
  primer_R
cctacacgacgctcttccgatctacagagccctcccacagagg

SEQ ID NO: 7 - DNA, 47 bp, artificial sequence
  primer_F
cctacacgacgctcttccgatcttgcaaaaattgaagttgtcagcac

SEQ ID NO: 8 - DNA, 44 bp, artificial sequence
  primer_R
cctacacgacgctcttccgatctaaggttggtaggggcctgatt

SEQ ID NO: 9 - DNA, 47 bp, artificial sequence
  primer_F
cctacacgacgctcttccgatcttcccaagattaattggtcctctca

SEQ ID NO: 10 - DNA, 47 bp, artificial sequence
  primer_R
cctacacgacgctcttccgatctacttaggatggacacaaagcagaa

SEQ ID NO: 11 - DNA, 43 bp, artificial sequence
  primer_F
cctacacgacgctcttccgatcttcctgggcctgcttcttcat

SEQ ID NO: 12 - DNA, 44 bp, artificial sequence
  primer_R
cctacacgacgctcttccgatctgccaagttcttacgggctggt

SEQ ID NO: 13 - DNA, 43 bp, artificial sequence
  primer_F
cctacacgacgctcttccgatctcctcctctccctctgcgttt

SEQ ID NO: 14 - DNA, 43 bp, artificial sequence
  primer_R
cctacacgacgctcttccgatctaaaggggcctgcaccattta

SEQ ID NO: 15 - DNA, 47 bp, artificial sequence
  primer_F
cctacacgacgctcttccgatctcctcatttgtcctgaccatgtctc

SEQ ID NO: 16 - DNA, 46 bp, artificial sequence
  primer_R
cctacacgacgctcttccgatctatctcagagtctcctggctggtg

SEQ ID NO: 17 - DNA, 47 bp, artificial sequence
  primer_F
cctacacgacgctcttccgatctgctggtgctctgctaagtatgctc

SEQ ID NO: 18 - DNA, 47 bp, artificial sequence
  primer_R
cctacacgacgctcttccgatctcaaaagatgttgaatgcctgaagc

SEQ ID NO: 19 - DNA, 43 bp, artificial sequence
  primer_F
cctacacgacgctcttccgatctccctcccctcgtttcctttc

SEQ ID NO: 20 - DNA, 42 bp, artificial sequence
  primer_R
cctacacgacgctcttccgatctctgtgctggggtcatggaa

SEQ ID NO: 21 - DNA, 43 bp, artificial sequence
  primer_F
cctacacgacgctcttccgatctttgtgtggaccaagccacct

SEQ ID NO: 22 - DNA, 44 bp, artificial sequence
  primer_R
cctacacgacgctcttccgatctttggggctctctaccatggac

SEQ ID NO: 23 - DNA, 44 bp, artificial sequence
  primer_F
cctacacgacgctcttccgatcttgccaagttcccaaggactaa

SEQ ID NO: 24 - DNA, 49 bp, artificial sequence
  primer_R
cctacacgacgctcttccgatctgccacctaggtaacatttagctacca

SEQ ID NO: 25 - DNA, 46 bp, artificial sequence
  primer_F
cctacacgacgctcttccgatctttcacaggccacatcaaaaagtc

SEQ ID NO: 26 - DNA, 53 bp, artificial sequence
  primer_R
cctacacgacgctcttccgatcttggcacctagattttatttacactttatct

SEQ ID NO: 27 - DNA, 45 bp, artificial sequence
  primer_F
cctacacgacgctcttccgatctaacagccatttgggtgaatttt

SEQ ID NO: 28 - DNA, 50 bp, artificial sequence
  primer_R
cctacacgacgctcttccgatctggattaaagaaaccattgtatgtcctg

SEQ ID NO: 29 - DNA, 47 bp, artificial sequence
  primer_F
cctacacgacgctcttccgatctaacaacataatgggattgttgagg

SEQ ID NO: 30 - DNA, 46 bp, artificial sequence
  primer_R
cctacacgacgctcttccgatctccctcaagaagaaaaggcattga

SEQ ID NO: 31 - DNA, 47 bp, artificial sequence
  primer_F
cctacacgacgctcttccgatctccctaacttccagatcaatcacac

SEQ ID NO: 32 - DNA, 49 bp, artificial sequence
  primer_R
cctacacgacgctcttccgatctggatcaggagactcaaacactacaat

SEQ ID NO: 33 - DNA, 43 bp, artificial sequence
  primer_F
cctacacgacgctcttccgatctggggccaaagtaggtgagga

SEQ ID NO: 34 - DNA, 47 bp, artificial sequence
  primer_R
cctacacgacgctcttccgatctcacctttcatcttcatggtcttcc

SEQ ID NO: 35 - DNA, 43 bp, artificial sequence
  primer_F
cctacacgacgctcttccgatctaggacccaggcagaatagcc

SEQ ID NO: 36 - DNA, 43 bp, artificial sequence
  primer_R
cctacacgacgctcttccgatctgctctggacgctagccatgt

SEQ ID NO: 37 - DNA, 46 bp, artificial sequence
  primer_F
cctacacgacgctcttccgatctcaatgaggcagtcatttgtgagc

SEQ ID NO: 38 - DNA, 50 bp, artificial sequence
  primer_R
cctacacgacgctcttccgatctcaacaaaacaagcatttatttctgcaa

SEQ ID NO: 39 - DNA, 47 bp, artificial sequence
  primer_F
cctacacgacgctcttccgatctggctgagagtcaaaccatcctttt

SEQ ID NO: 40 - DNA, 45 bp, artificial sequence
  primer_R
cctacacgacgctcttccgatctggggtccctctacaggaacatt

SEQ ID NO: 41 - DNA, 46 bp, artificial sequence
  primer_F
cctacacgacgctcttccgatcttccattctttggttggttcctca

SEQ ID NO: 42 - DNA, 46 bp, artificial sequence
  primer_R
cctacacgacgctcttccgatctgagctgcatgaaatttggagctt

SEQ ID NO: 43 - DNA, 44 bp, artificial sequence
  primer_F
cctacacgacgctcttccgatctaacacagctcagtcgtggttg

SEQ ID NO: 44 - DNA, 43 bp, artificial sequence
  primer_R
cctacacgacgctcttccgatcttgagctccttggcattcagg

SEQ ID NO: 45 - DNA, 43 bp, artificial sequence
  primer_F
cctacacgacgctcttccgatcttgtggcagtggctgcaatag

SEQ ID NO: 46 - DNA, 45 bp, artificial sequence
  primer_R
cctacacgacgctcttccgatctaaagccagagccctttcttgtc

SEQ ID NO: 47 - DNA, 49 bp, artificial sequence
  primer_F
cctacacgacgctcttccgatcttctgggtttatctacctcagaaggtg

SEQ ID NO: 48 - DNA, 48 bp, artificial sequence
  primer_R
cctacacgacgctcttccgatctcaaaaatgaaatggattcatcagga

SEQ ID NO: 49 - DNA, 44 bp, artificial sequence
  primer_F
cctacacgacgctcttccgatctcgacaggatgtcgtcggagat

SEQ ID NO: 50 - DNA, 46 bp, artificial sequence
  primer_R
cctacacgacgctcttccgatctctcccaccccagaacatctcatc

SEQ ID NO: 51 - DNA, 45 bp, artificial sequence
  primer_F
cctacacgacgctcttccgatctggctgtaggctccgcatctgta

SEQ ID NO: 52 - DNA, 43 bp, artificial sequence
  primer_R
cctacacgacgctcttccgatctcagctgcagtccgtgggtct

SEQ ID NO: 53 - DNA, 46 bp, artificial sequence
  primer_F
cctacacgacgctcttccgatctcctgccaagaaacagagacacaa

SEQ ID NO: 54 - DNA, 46 bp, artificial sequence
  primer_R
cctacacgacgctcttccgatctggacacttacatccccatcttgg

SEQ ID NO: 55 - DNA, 46 bp, artificial sequence
  primer_F
cctacacgacgctcttccgatcttcctgatcactcagcctgaagac

SEQ ID NO: 56 - DNA, 43 bp, artificial sequence
  primer_R
cctacacgacgctcttccgatcttgaagggtatgcctgtcgtg

SEQ ID NO: 57 - DNA, 47 bp, artificial sequence
  primer_F
cctacacgacgctcttccgatctccaagaaggataactgatggactc

SEQ ID NO: 58 - DNA, 36 bp, artificial sequence
  primer_R
gacgctcttccgatctagcagttcctgcttgccatg

SEQ ID NO: 59 - DNA, 50 bp, artificial sequence
  primer_F
cctacacgacgctcttccgatcttgataatctaattttgtttgtgctcca

SEQ ID NO: 60 - DNA, 43 bp, artificial sequence
  primer_R
cctacacgacgctcttccgatctagcagctgggacctttttgc

SEQ ID NO: 61 - DNA, 47 bp, artificial sequence
  primer_F
cctacacgacgctcttccgatctttgatattcatcttggcacccata

SEQ ID NO: 62 - DNA, 49 bp, artificial sequence
  primer_R
cctacacgacgctcttccgatctcagcagttttattctcttcactgcaa

SEQ ID NO: 63 - DNA, 45 bp, artificial sequence
  primer_F
cctacacgacgctcttccgatctttcacgcactttcagaagtcct

SEQ ID NO: 64 - DNA, 51 bp, artificial sequence
  primer_R
cctacacgacgctcttccgatctttcatttactggattcaagttacagtcc

SEQ ID NO: 65 - DNA, 48 bp, artificial sequence
  primer_F
cctacacgacgctcttccgatctgtgctgaaacagagacaggtaaaaa

SEQ ID NO: 66 - DNA, 52 bp, artificial sequence
  primer_R
cctacacgacgctcttccgatctcctgatatcactactgcagacaaactaat

SEQ ID NO: 67 - DNA, 46 bp, artificial sequence
  primer_F
cctacacgacgctcttccgatctcactctgacagtgaggttcttgg

SEQ ID NO: 68 - DNA, 45 bp, artificial sequence
  primer_R
cctacacgacgctcttccgatctagggcatgtcattgcctcaaag

SEQ ID NO: 69 - DNA, 43 bp, artificial sequence
  primer_F
cctacacgacgctcttccgatctactcccccgaaatgaacagc

SEQ ID NO: 70 - DNA, 43 bp, artificial sequence
  primer_R
cctacacgacgctcttccgatctcctggagagcgccatctctg

SEQ ID NO: 71 - DNA, 45 bp, artificial sequence
  primer_F
cctacacgacgctcttccgatcttggagacgcagagaagagaacg

SEQ ID NO: 72 - DNA, 50 bp, artificial sequence
  primer_R
cctacacgacgctcttccgatctcattcgggtagaacgttgtattacagt

SEQ ID NO: 73 - DNA, 43 bp, artificial sequence
  primer_F
cctacacgacgctcttccgatctgcagggaaaaggctcagtcc

SEQ ID NO: 74 - DNA, 46 bp, artificial sequence
  primer_R
cctacacgacgctcttccgatcttccagagctttgcagtgttcttc

SEQ ID NO: 75 - DNA, 43 bp, artificial sequence
  primer_F
cctacacgacgctcttccgatctatgggccaggttttgagctg

SEQ ID NO: 76 - DNA, 43 bp, artificial sequence
  primer_R
cctacacgacgctcttccgatctcatggaggtgacgggctatc

SEQ ID NO: 77 - DNA, 43 bp, artificial sequence
  primer_F
cctacacgacgctcttccgatcttccttgggcctgagaagacc

SEQ ID NO: 78 - DNA, 43 bp, artificial sequence
  primer_R
cctacacgacgctcttccgatctaggatccactccccctaccc

SEQ ID NO: 79 - DNA, 44 bp, artificial sequence
  primer_F
cctacacgacgctcttccgatctatggggagtgggtgggtaatg

SEQ ID NO: 80 - DNA, 46 bp, artificial sequence
  primer_R
cctacacgacgctcttccgatcttgggattccacgtatgtgtttgc

SEQ ID NO: 81 - DNA, 46 bp, artificial sequence
  primer_F
cctacacgacgctcttccgatctctgctggataacttggaggtgct

SEQ ID NO: 82 - DNA, 48 bp, artificial sequence
  primer_R
cctacacgacgctcttccgatcttgcatatactgcagagacaagcaaa

SEQ ID NO: 83 - DNA, 47 bp, artificial sequence
  primer_F
cctacacgacgctcttccgatctcattttcagctcctttcaaacctg

SEQ ID NO: 84 - DNA, 44 bp, artificial sequence
  primer_R
cctacacgacgctcttccgatcttgggcaatcattttgaaccaa

SEQ ID NO: 85 - DNA, 44 bp, artificial sequence
  primer_F
cctacacgacgctcttccgatctccccctggaattttgtaaagc

SEQ ID NO: 86 - DNA, 43 bp, artificial sequence
  primer_R
cctacacgacgctcttccgatctgcccggttcttggagatgag

SEQ ID NO: 87 - DNA, 46 bp, artificial sequence
  primer_F
cctacacgacgctcttccgatctgacgacacctccaggtgcattag

SEQ ID NO: 88 - DNA, 47 bp, artificial sequence
  primer_R
cctacacgacgctcttccgatctttcaacagatggagcaaagcctta

SEQ ID NO: 89 - DNA, 43 bp, artificial sequence
  primer_F
cctacacgacgctcttccgatctctcaggcccagggcttactc

SEQ ID NO: 90 - DNA, 43 bp, artificial sequence
  primer_R
cctacacgacgctcttccgatcttaacaaggcagccagaagca

SEQ ID NO: 91 - DNA, 45 bp, artificial sequence
  primer_F
cctacacgacgctcttccgatctggaacagggaaagtcagtggtg

SEQ ID NO: 92 - DNA, 43 bp, artificial sequence
  primer_R
cctacacgacgctcttccgatcttgtgcctggccaagagatac

SEQ ID NO: 93 - DNA, 45 bp, artificial sequence
  Adapter Universal Primer F
tcagacgtgtgctcttccgatctcaagaacggaatgtgtacttgc

SEQ ID NO: 94 - DNA, 45 bp, artificial sequence
  Adapter Universal Primer R
tcagacgtgtgctcttccgatctctctcgctaacaagctcagcta

SEQ ID NO: 95 - DNA, 45 bp, artificial sequence
  UNIPCRF
aatgatacggcgaccaccgagatctacactctttccctacacgac

SEQ ID NO: 96 - DNA, 52 bp, artificial sequence
  UDIR0001
caagcagaagacggcatacgagataaccgcgggtgactggagttcagacgtg

SEQ ID NO: 97 - DNA, 52 bp, artificial sequence
  UDIR0002
caagcagaagacggcatacgagatggttataagtgactggagttcagacgtg

SEQ ID NO: 98 - DNA, 52 bp, artificial sequence
  UDIR0003
caagcagaagacggcatacgagatccaagtccgtgactggagttcagacgtg